(19) United States
(12) Patent Application Publication
(10) Pub. No.: US 2011/0264067 A1
(43) Pub. Date: Oct. 27, 2011

(54) Feminine hygiene pads

(76) Inventor: Ilse Rubio, Los Angeles, CA (US)

(21) Appl. No.: 12/803,334

(22) Filed: Jun. 24, 2010

Related U.S. Application Data
(63) Continuation-in-part of application No. 29/349,696, filed on Apr. 23, 2010, now Pat. No. D626219.

(51) Int. Cl. A61F 13/15

(52) U.S. Cl. 604/385.04

(57) ABSTRACT

Feminine hygiene pads are provided that include an elongate main pad portion including an intermediate region between forward and rear regions; and a pair of flaps extending laterally from the intermediate region, wherein at least a portion of the intermediate region and the flaps include material having a color for concealing bodily fluids staining the intermediate region and flaps, e.g., black or other non-white color. For example, the color may be similar to the color of an undergarment to which the pad is secured. Optionally, each flap may include tapered side edges and/or may include an outer edge that includes a plurality of arcuate lobes separated by scallops.

### RELATED APPLICATION DATA

This application claims priority to and is a continuation-in-part of co-pending design application Ser. No. 29/349, 696 filed Apr. 23, 2010, the entire disclosure of which is expressly incorporated by reference herein.

### FIELD OF THE INVENTION

The present invention generally relates to feminine hygiene pads and, more particularly, to feminine hygiene pads including color wings or flaps, and to methods for making and using such pads.

### BACKGROUND

Feminine hygiene pads are disposable, liquid absorbent pads applied to the crotch area by women in order to absorb menstrual fluids, urine, and/or other substances. Existing feminine hygiene pads are generally elongate pads that may include lateral flaps or wings, e.g., which may be wrapped around a panty or other garment to help secure the pad thereto. Generally, pads are formed from white or light colored materials. However, if the pad leaks fluid, the resulting stain may be prominently visible.

Accordingly, improved feminine hygiene pads would be useful.

### SUMMARY

The present invention is directed generally to feminine hygiene pads and, more particularly, to absorbent and disposable feminine hygiene pads including color wings or flaps, and to methods for making and using such pads.

In accordance with one embodiment, a feminine hygiene pad is provided that includes an elongate main pad portion including an intermediate region between forward and rear regions; and a pair of flaps extending laterally from the intermediate region, wherein at least a portion of the intermediate region and the flaps include material having a color for at least partially concealing bodily fluids staining the intermediate region and flaps. For example, the color may be black or other non-white, relatively dark color, e.g., similar to an undergarment to which the pad is to be attached and/or to fluids that may be captured by the pad. Optionally, an entire upper surface and/or lower surface of each flap and/or the intermediate region may include the color.

In an exemplary embodiment, the main pad portion may include a central pad portion and a relatively thin periphery surrounding the central pad portion. In this embodiment, the flaps may have a different color than the central pad portion and/or the periphery adjacent the flaps may have a different color than the central pad portion. For example, the central pad portion may be formed from white, or other light colored material and the flaps and/or periphery adjacent the flaps may be formed from relatively darker colored material, e.g., for concealing stains and/or matching an undergarment with which the pad is worn.

In one embodiment, each flap may include side edges extending outwardly from the intermediate region and an outer edge extending between the side edges. Optionally, the outer edge may include a plurality of arcuate lobes separated by scallops or other decorative features. In addition or alternatively, the side edges of each flap may taper away from the intermediate region such that the outer edge of the flap has a shorter length than a base of the flap along the intermediate region.

In accordance with another embodiment, the combination of a feminine hygiene pad and an undergarment may be provided, the undergarment including a color. The pad may include an elongate main pad portion including an intermediate region between forward and rear regions; and a pair of flaps extending laterally from the intermediate region, wherein at least a portion of the intermediate region and the flaps comprise material having a color similar to the color of the undergarment. For example, the colors of the pad and the undergarment may be black or other non-white colors, e.g., that may conceal blood or stains from other bodily fluids.

In accordance with still another embodiment, a feminine hygiene pad is provided for use with an undergarment having a color. Generally, the pad may include an elongate main pad portion including an intermediate region between forward and rear regions; a pair of flaps extending laterally from the intermediate region, each flap including side edges extending outwardly from the intermediate region and an outer edge extending between the side edges, wherein the outer edge include a plurality of arcuate lobes separated by scallops or other decorative features. The main portion and the flaps may include adhesive regions for securing the pad to the undergarment, and at least a portion of the intermediate region and the flaps may include material having a color similar to the undergarment for at least partially concealing bodily fluids staining the intermediate region and flaps.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of an exemplary embodiment of a feminine hygiene pad.

FIG. 2 is a top view of the pad of FIG. 1 showing an exemplary color pattern on an intermediate region of the pad.

FIG. 3 is a bottom view of the pad of FIG. 1.

FIGS. 4 and 5 are side views of the pad of FIG. 1.

FIGS. 6A and 6B are front and bottom views, respectively, showing an undergarment to which the pad of FIGS. 1-5 is attached.

FIGS. 7A-7F are top views of alternative shapes that may be provided for the pad of FIGS. 1-5.

### DETAILED DESCRIPTION

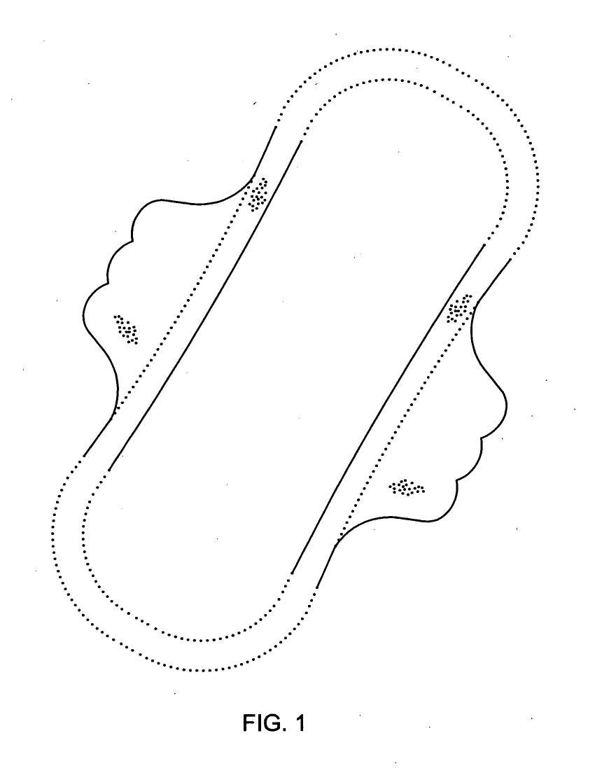

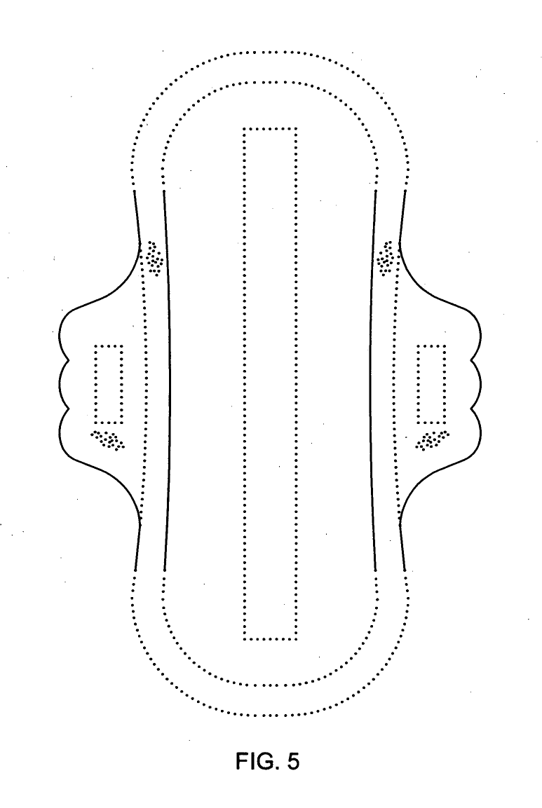

Turning to the drawings, FIGS. 1-5 show an exemplary embodiment of a feminine hygiene pad 10 that generally includes an elongate main pad portion 20 including an intermediate region 24 between a forward region 22 and a rear region 26, and a pair of flaps or wings 30 extending laterally from the intermediate region 24. As explained further below, at least a portion of the intermediate region 24 and/or the flaps 30 may include material having a desired color 37 (represented generally by the stippling on FIGS. 1 and 3), e.g., similar to an undergarment to which the pad 10 is intended to be secured and/or for at least partially concealing bodily fluids staining the intermediate region 24 and/or flaps 30, and/or for providing a desired uniform or clean appearance.

As best seen in FIGS. 1-3, the main portion 20 of the pad 10 may include a central pad portion 12 extending between the forward and rear regions 22, 26 and an outer periphery 14 surrounding the central pad portion 12. For example, the central pad portion 12 may include one or more relatively thick, absorbent inner layers (not shown) surrounded by the outer periphery 14, and one or more relatively thin outer layers, which may extend over the outer periphery 14. For example, the outer layer on an upper surface 16 and/or lower surface 18 of the pad 10 may include porous material, e.g., which may allow blood, urine, other substances, or other bodily fluids passing there through to be absorbed or otherwise retained by the one or more inner layers.

As shown, the main portion 12 may have a generally elongate rounded shape, e.g., substantially symmetrical between the front and rear regions 22, 26. Alternatively, the front and rear regions 22, 26 may have different shapes, e.g., the rear region 26 may have a tapered or other shape, e.g., similar to the pads disclosed in U.S. Pat. No. 7,037,297 issued May 2, 2006, the entire disclosure of which is expressly incorporated by reference herein. In exemplary embodiments, the pad 10 may be a sanitary napkin, a pantiliner, or an incontinence pad. In addition or alternatively, the flaps 30 may have a variety of lengths and/or widths, as desired. Generally, the flaps 30 may be proportional to the length of the main pad portion 20, e.g., such that the flaps 30 coincide with the intermediate region 24, although, alternatively, the flaps 30 may extend and/or taper gradually between the forward and/or rear regions 22, 26 of the main pad portion 20, if desired.

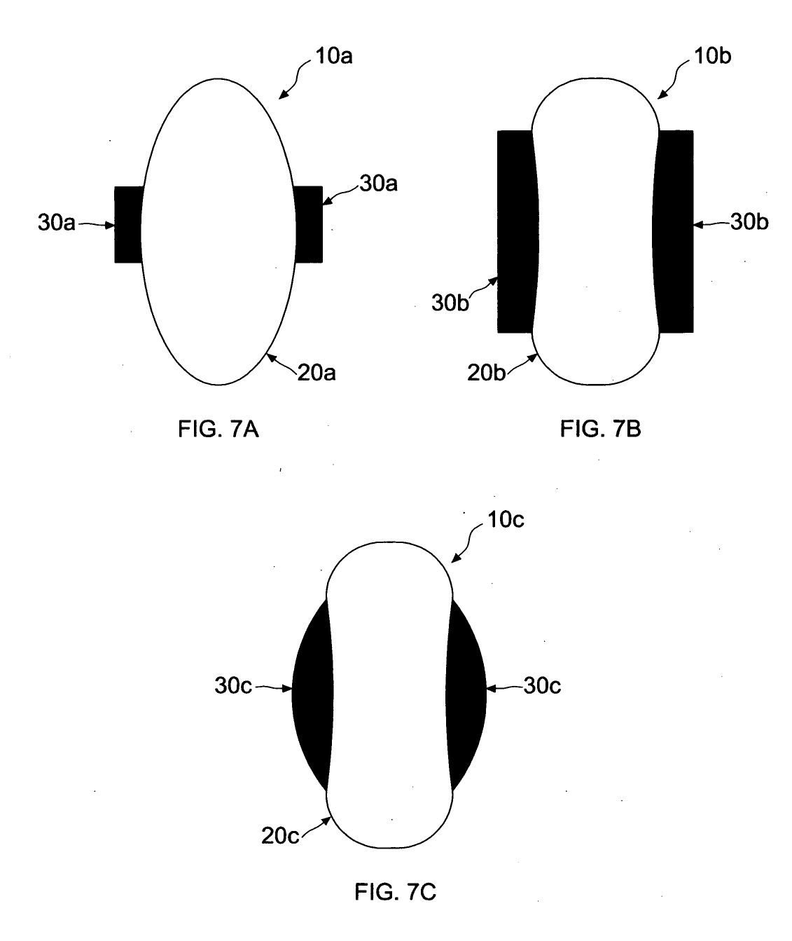

For example, FIGS. 7A-7F show alternative shapes for the main portion and/or the flaps or wings that may be included in any of the embodiments herein. FIG. 7A shows an exemplary embodiment of a pad 10a in which the main pad portion 20a is generally elliptical and the flaps 30a are generally rectangular. The pad 10b of FIG. 7B is an elongated partially rounded ends generally rectangular similar to the pad 10a of FIG. 7A, except that the flaps 30b have a greater width, e.g., extending to the forward and rear regions. FIG. 7C shows a pad 10c including a an elongated partially rounded ends generally rectangular pad portion 20c and curved flaps 30c extending longitudinally along the side edges thereof. FIG. 7D shows an exemplary embodiment of a pad 10d including a main pad portion 20d that includes an elongated relatively narrow tail portion 28d and a pair of generally rectangular flaps 30d.

Turning to FIG. 7E, another pad 10e is shown that includes a main pad portion 20e having a generally tapered or tear shape and a pair of flaps 30e extending laterally from an intermediate region of the main pad portion 20e. FIG. 7F shows yet another embodiment of a pad 10f including a main pad portion 20f and a paid of flaps 30f extending from the main pad portion 20f. Unlike other embodiments, the pad 10f may be foldable, e.g., to change the size and/or shape, as desired, e.g., similar to the pads shown in co-pending application Ser. No. 11/811,497, filed Jun. 11, 2007, and published as U.S. Publication No. 2008/0009818, the entire disclosure of which is expressly incorporated by reference herein. It will be appreciated that the various shapes of the main pad portion 20s-20f and/or flaps 30a-30f may be interchanged in any of the embodiments shown herein to provide a desired combination.

Returning to FIGS. 1-3, each flap 30 may include side edges 32 extending outwardly from the intermediate region 24 and an outer edge 34 extending between the side edges 32. Optionally, the outer edge 34 may include one or more decorative features, for example, a plurality of arcuate lobes 36 (three shown) separated by scallops 38 (two shown), as shown. In addition or alternatively, the side edges 32 of each flap 30 may taper away from the intermediate region 24, e.g., such that the outer edge 34 of the flap 30 has a shorter length than a base 31 of the flap along the intermediate region 24. Alternatively, as shown in FIGS. 7A, 7B, and 7D, the flaps may have substantially straight side edges. It will be appreciated that the base 31 of the flaps 30 may be integrally formed with or otherwise attached to the intermediate region 24 of the main pad portion 20, as represented by the dashed lines along the base 31.

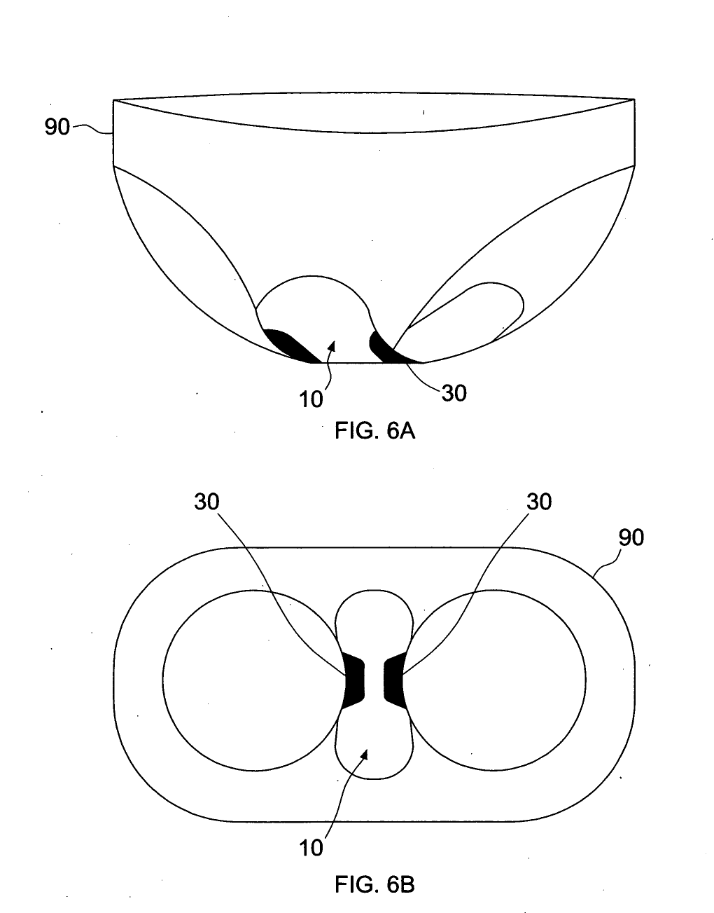

As shown in FIG. 3, one or more adhesive regions 40a, 40b may be provided on a lower surface of the pad 10, e.g., for securing the pad 10 to an undergarment 90, e.g., as shown in FIGS. 6A and 6B and described further below. For example, the main portion 20 may include a single adhesive region 40a extending longitudinally between the forward and rear regions 22, 26 or, alternatively, multiple adhesive regions (not shown), e.g., on the forward and rear regions 22. In addition, each flap 30 may include an adhesive region 40b, e.g., to secure the flap 30 around a portion of an undergarment. In an exemplary embodiment, each adhesive region 40 may include double sided tape with an inner surface substantially permanently attached to the outer layer of the pad 10 and an outer surface covered with a removable sheet (not shown). The cover sheet may be removed to expose a relatively low tack adhesive on the outer surface that may be used to removably secure the pad 10 to an undergarment.

As can be seen in FIG. 1, at least a portion of the upper surface of the pad 10 may be formed from material having a color 37 for concealing stains, to have a clean appearance and/or matching an undergarment, e.g., black, purple, green, blue, red, or other non-white color, e.g., contrasting or otherwise darker than the color of the rest of the pad 10. For example, in one embodiment, the outer layer of the entire upper surface of the main pad portion 20 and the flaps 30 may be formed from material having such color. Optionally, the lower surface of the main pad portion 20 and/or flaps 30 may also include such a color.

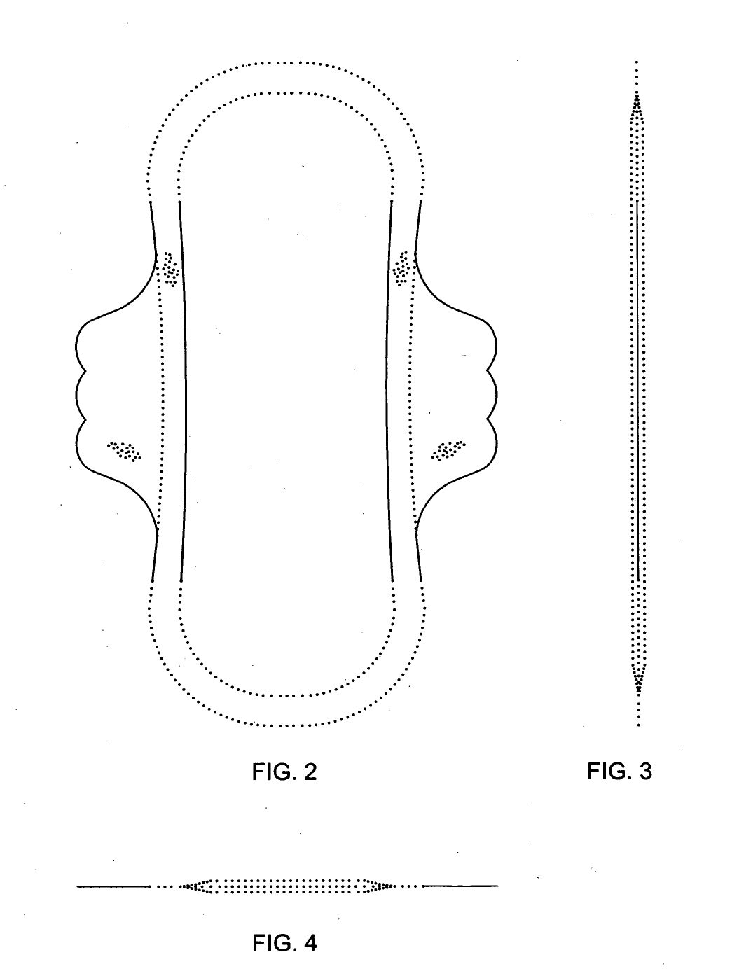

Alternatively, only a portion of the intermediate region 24 of the main pad portion 20 and/or the flaps 30 may include such color. For example, in the embodiment shown in FIG. 2, the upper surface of the outer periphery 14 of the intermediate region 24 may include a black or other concealing color, as well as the upper surfaces and/or lower surfaces of the flaps 30. Thus, the colored regions may contrast with the white, or relatively lighter color of the central pad portion 12 and/or periphery 14 of the forward and rear regions 22, 26.

The length of the colored region of the outer periphery 14 of the intermediate region 24 may be substantially the same as the width of the flaps 30, may be longer (e.g., as shown in FIG. 2), or may be shorter than the width of the flaps 30, as desired. Since these regions are closest to the central pad portion 12, they are more likely to be exposed to any leaks. Thus, in this alternative, the remaining areas of the pad 10 may be white, or other relatively lighter color, as desired.

In a further alternative, only the flaps 30 may include the color 37, e.g., on one of the upper surface (shown in FIGS. 1 and 2), the lower surface (shown in FIG. 3), or both. In this alternative, the entire main pad portion 20 may be white, or other relatively lighter color. For example, only the upper surface of the flaps 30 may include the color 37 such that when the flaps 30 are folded around an undergarment 90 (as described below), only the upper surface of the flaps 30 including the color 37 may be visible when the undergarment 90 is worn.

Turning to FIGS. 6A and 6B, during use, a pad 10 may be selected having a desired color in at least the intermediate region 24 and/or flaps 30. For example, the user may be wearing a pair of panties or other undergarment 90 having a desired color, and a pad 10 may be selected having a similar color in the intermediate region 24 and/or flaps 30 to the undergarment 90. Any cover sheets on the adhesive regions 40 may be removed, and the lower surface of the main pad portion 12 may be applied to the undergarment 90 to secure the adhesive regions 40a to an inner surface of the undergarment 90. Simultaneously or subsequently, as shown in FIG. 6A, the flaps 30 may be wrapped around the undergarment 90 and secured to an underside of the undergarment by the adhesive regions 40b, as shown in FIG. 6B.

The similar colors of the undergarment 90 and/or pad 10 may conceal the presence of the pad 10. In addition, the color 37 of the pad 10 may conceal any bodily fluids, e.g., if they leak from the central pad portion 12 of the pad 10. In particular, the outer periphery 14 of the intermediate region 24 and the flaps 30 may be closest to the central pad portion 12 and, therefore may be more likely to become stained if the pad 10 leaks. However, if the material of the intermediate region 24 and/or flaps 30 include a dark or other non-white color, the material may conceal the stain, which may reduce embarrassment and/or discomfort, providing a clean appearance

It will be appreciated that elements or components shown with any embodiment herein are exemplary for the specific embodiment and may be used on or in combination with other embodiments disclosed herein.

While embodiments of the present invention have been shown and described, various modifications may be made without departing from the scope of the present invention. The invention, therefore, should not be limited, except to the following claims, and their equivalents.

I claim:

1. A feminine hygiene pad, comprising:
an elongate main pad portion including an intermediate region between forward and rear regions; and
a pair of flaps extending laterally from the intermediate region,
wherein the flaps comprise material having a color for at least partially concealing bodily fluids staining the flaps.

2. The pad of claim 1, wherein one or more regions of the main pad portion comprise material having a color different than the color of the material of the flaps.

3. The pad of claim 1, wherein at least a portion of the intermediate region comprises the color.

4. The pad of claim 3, wherein the color is similar to the color of the undergarment.

5. The pad of claim 1, wherein each flap comprises side edges extending outwardly from the intermediate region and an outer edge extending between the side edges, wherein the outer edge comprises a plurality of arcuate lobes separated by scallops.

6. The pad of claim 5, wherein the side edges of each flap taper away from the intermediate region such that the outer edge of the flap has a shorter length than a base of the flap along the intermediate region.

7. The pad of claim 1, wherein each flap has an upper surface and a lower surface, and wherein at least one of the entire upper surface and the entire lower surface include the color.

8. The pad of claim 1, wherein the main portion comprises a central pad portion extending between the forward and rear regions and an outer periphery surrounding the central pad portion, and wherein the outer periphery of the intermediate region comprises the color.

9. The pad of claim 8, wherein the central pad portion comprises a relatively light color compared to the color.

10. The pad of claim 1, wherein the pad comprises one of a sanitary napkin, a pantiliner, and an incontinence pad.

11. The combination of a feminine hygiene pad and an undergarment, the undergarment comprising a color, the pad comprising:
an elongate main pad portion including an intermediate region between forward and rear regions; and
a pair of flaps extending laterally from the intermediate region,
wherein the flaps comprise material having a non-white or color similar to the color of the undergarment.

12. The combination of claim 11, wherein the colors of the pad and the undergarment are sufficiently dark for at least partially concealing bodily fluids staining the flaps.

13. The combination of claim 11, wherein each flap comprises side edges extending outwardly from the intermediate region and an outer edge extending between the side edges, wherein the outer edge comprises a plurality of arcuate lobes separated by scallops.

14. The combination of claim 11, wherein the side edges of each flap taper away from the intermediate region such that the outer edge of the flap has a shorter length than a base of the flap along the intermediate region.

15. The combination of claim 14, wherein each flap has an upper surface and a lower surface, and wherein at least one of the entire upper surface and the entire lower surface include the color.

16. The combination of claim 11, wherein the main portion comprises a central pad portion extending between the forward and rear regions and an outer periphery surrounding the central pad portion, and wherein the outer periphery of the intermediate region comprises the color.

17. The combination of claim 11, wherein the pad comprises one of a sanitary napkin, a pantiliner, and an incontinence pad.

18. A feminine hygiene pad for use with an undergarment having a color, the pad comprising:
an elongate main pad portion including an intermediate region between forward and rear regions;
a pair of flaps extending laterally from the intermediate region;
the main portion and the flaps comprising adhesive regions on a lower surface thereof for securing the pad to the undergarment,
wherein at least an upper surface of the flaps comprises a color similar to the undergarment for at least partially concealing bodily fluids staining the flaps, and
wherein one or more regions of the main pad portion comprise a color different than the color of the upper surface of the flaps.

19. The pad of claim 18 wherein at least a lower surface of the intermediate region comprises the same color as the upper surface of the flaps.

20. The pad of claim 19, wherein the one or more regions of the main pad portion comprise a relatively light color compared to the upper surface of the flaps.
